SEQUENCE LISTING (26 sequences)

SEQ ID NO: 1 - protein, 406 aa, Homo sapiens
  misc_feature (7)..(7): Xaa is 4-carboxyglutamic acid
ANAFLGXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 35 aa, Homo sapiens
AEAVFPDVDYVNSTEAETILDNITQSTQSFNDFTR

SEQ ID NO: 3 - protein, 52 aa, Homo sapiens
SVAQATSSSGEAPDSITWKPYDAADLDPTENPFDLLDFNQTQPERGDNNLTR

SEQ ID NO: 4 - DNA, 24 bp, Artificial
  PCR primer A
tcactataggctagcatggtctcc

SEQ ID NO: 5 - DNA, 41 bp, Artificial
  PCR primer B_C
cttggtcttcttggtcttctgtgtcgccttggggtttgctg

SEQ ID NO: 6 - DNA, 39 bp, Artificial
  PCR primer E_C
accaagaagaccaagtagatccgcgaattgtggggggca

SEQ ID NO: 7 - DNA, 24 bp, Artificial
  PCR primer B_IX
aaacagtctcagcgccttggggtt

SEQ ID NO: 8 - DNA, 24 bp, Artificial
  PCR primer C_IX
aaccccaaggcgctgagactgttt

SEQ ID NO: 9 - DNA, 23 bp, Artificial
  PCR primer D_IX
ccccacaattcgagtgaagtcat

SEQ ID NO: 10 - DNA, 23 bp, Artificial
  PCR primer E_IX
atgacttcactcgaattgtgggg

SEQ ID NO: 11 - DNA, 23 bp, Artificial
  PCR primer B_X
tgggccactgagccttggggttt

SEQ ID NO: 12 - DNA, 23 bp, Artificial
  PCR primer C_X
aaaccccaaggctcagtggccca

SEQ ID NO: 13 - DNA, 24 bp, Artificial
  PCR primer D_X
ccccacaattcgggtgaggttgtt

SEQ ID NO: 14 - DNA, 24 bp, Artificial
  PCR primer E_X
aacaacctcacccgaattgtgggg

SEQ ID NO: 15 - DNA, 24 bp, Artificial
  PCR primer D_X1-34
ccacaattcggtcgaaggggttct

SEQ ID NO: 16 - DNA, 24 bp, Artificial
  PCR primer E_X1-34
agaaccccttcgaccgaattgtgg

SEQ ID NO: 17 - DNA, 23 bp, Artificial
  PCR primer B_X30-52
aaggggttctcgccttggggttt

SEQ ID NO: 18 - DNA, 23 bp, Artificial
  PCR primer C_X30-52
aaaccccaaggcgagaacccctt

SEQ ID NO: 19 - DNA, 27 bp, Artificial
  PCR primer F
taaagggaagcggccgcctagggaaat

SEQ ID NO: 20 - DNA, 51 bp, Artificial
  PCR primer NNAAfwd
gcttgacttcgcccagacgcagcctgagaggggcgacaacgccctcacccg

SEQ ID NO: 21 - DNA, 51 bp, Artificial
  PCR primer NNAArev
cgggtgagggcgttgtcgcccctctcaggctgcgtctgggcgaagtcaagc

SEQ ID NO: 22 - DNA, 55 bp, Artificial
  PCR primer TTAAfwd
gacagcatcgcatggaagccatatgatgcagccgacctggaccccgccgagaacc

SEQ ID NO: 23 - DNA, 55 bp, Artificial
  PCR primer TTAArev
ggttctcggcggggtccaggtcggctgcatcatatggcttccatgcgatgctgtc

SEQ ID NO: 24 - DNA, 21 bp, Artificial
  PCR primer B_XHC
ctgggccactgagggaaatgg

SEQ ID NO: 25 - DNA, 21 bp, Artificial
  PCR primer C_XHC
ccatttccctcagtggcccag

SEQ ID NO: 26 - DNA, 30 bp, Artificial
  PCR primer D_XHC
taaagggaagcggccgcctaggtgaggttg

(19) United States
(12) Patent Application Publication
(10) Pub. No.: US 2012/0321607 A1
(43) Pub. Date: Dec. 20, 2012

(54) Factor VII Fusion Polypeptides

(75) Inventor: Egon Persson, Ballerup (DK)

(73) Assignee: Novo Nordisk Health Care AG, Zurich (CH)

(21) Appl. No.: 13/574,896

(22) PCT Filed: Jan. 27, 2011
(86) PCT No.: PCT/EP2011/051139

§ 371 Date: Sep. 4, 2012

Related U.S. Application Data
(60) Provisional application No. 61/302,723, filed on Feb. 9, 2010.

(30) Foreign Application Priority Data
Jan. 28, 2010 (EP) 10151940.3

(51) Int. Cl. C12N 9/96; C12N 15/63; C12N 1/19; A61P 7/04; C12N 5/10; C12N 15/62; A61K 38/48

(52) U.S. Cl. 424/94.3; 435/188; 536/23.2; 435/320.1; 435/325; 435/358; 435/352; 435/369; 435/254.2; 435/254.21; 435/254.23

(57) ABSTRACT

The present invention relates to a Factor VII (FVII) fusion polypeptide with a prolonged half-life, wherein the FVII polypeptide can be activated or is in the activated form.

The present invention relates to a Factor VII (FVII) fusion polypeptide with a prolonged half-life, wherein the FVII polypeptide can be activated or is in the activated form.

FVII is a glycoprotein produced by the liver. The activated form of FVII, known as FVIIa, plays a central role in blood coagulation. Blood coagulation is achieved as a result of cascading interactions between a number of trypsin-like serine proteases. Many of these enzymes are synthesized as inactive precursor zymogens that are cleaved during limited proteolysis to generate their active forms.

In particular, FVII is activated to FVIIa, which binds to tissue factor, a lipoprotein constitutively expressed on the membrane of certain cells. Certain proteases, including FXa and FIXa, are known to cause activation of FVII to FVIIa. Activation of FVII involves the conversion of single chain FVII into an activated two-chain form (FVIIa) through proteolytic cleavage of the peptide bond between Arg(152) and Ile(153). The formation of a salt bridge between Ile(153) and Asp(343) drives the conversion of FVIIa from being zymogen-like to the active form.

When a blood vessel is damaged, tissue factor is exposed to blood and circulating FVIIa. The bound tissue factor and FVIIa then activates Factor IX (FIX) and Factor X (FX) to form FIXa and FXa, respectively.

Patients with haemophilia have little or no functional coagulation Factor VIII or Factor IX and some patients develop neutralising antibodies to the administered factor and require treatment with a bypassing agent such as Factor VIIa.

Optimally, treatment of haemophilia is prophylactic although prophylaxis with Factor FVIIa is currently cumbersome and expensive due to its short half-life. Repeated dosing is required in order to maintain the plasma concentration of Factor VIIa above a threshold level for the required time periods.

Modified FVII polypeptides have been developed to attempt to address the issue of short half-life. For example, WO2006/018204 describes FVII polypeptides that have been modified by inserting the activation peptide from FX or FIX between amino acids 140 to 152 of the FVII polypeptide sequence. However, it is not possible to activate the modified polypeptides described in WO2006/018204 to FVIIa.

Thus, there is an ongoing requirement for approaches to increase the half-life of FVII and FVIIa, while retaining the ability of FVII activation to FVIIa.

The present inventor has identified that by adding the activation peptide sequence from FX or from FIX into a certain position of a FVII polypeptide, a fusion FVII polypeptide is generated that can be activated to FVIIa, wherein both the unactivated FVII polypeptide and the activated FVII (FVIIa) polypeptide have an extended half-life.

The first aspect of the present invention relates to a polypeptide comprising a FVII or a FVIIa polypeptide or a homologue thereof and a FX or a FIX activation peptide or a homologue thereof, wherein the FX or FIX activation peptide is fused at the C-terminal of the FVII or FVIIa polypeptide.

The homologues of the FVII/FVIIa polypeptides and the FX/FIX activation peptides are biologically active homologues. “Biological activity” of FVII/FVIIa polypeptide homologues and activation peptide homologues is defined below.

The term “FVII polypeptide” as used herein refers to the mature unactivated FVII polypeptide.

The term “FVIIa polypeptide” as used herein refers to the activated form of the FVII polypeptide i.e. the FVII polypeptide cleaved at a single peptide bond at Arg152-Ile153 resulting in the formation of two polypeptide chains that are held together by a disulfide bridge. Thus, a FVIIa polypeptide has the same sequence as a FVII polypeptide but is formed of two polypeptide chains rather than one.

The FVII or FVIIa polypeptide of the invention may be truncated and the FX or FIX activation peptide is then fused at the C-terminal of the truncated polypeptide.

The term “truncated FVII or FVIIa polypeptide” as used herein includes a FVII or FVIIa polypeptide that does not comprise the first 1, 2, 3, 4, 5, 6, 7, 8, 9 or 10 amino acid residues at the C-terminal of the wild-type or homologue FVII or FVIIa polypeptide. A truncated FVII or FVIIa polypeptide according to the invention is biologically active.

The FVII or FVIIa polypeptide of the invention may also be extended and the FX or FIX activation peptide is then fused at the C-terminal of the extended polypeptide. The term “extended FVII or FVIIa polypeptide” as used herein includes to a FVII or FVIIa polypeptide that additionally comprises 1, 2, 3, 4, 5, 6, 7, 8, 9, 10 or more amino acid residues at the C-terminal of the wild-type or homologous FVII or FVIIa polypeptide. An extended FVII or FVIIa polypeptide according to the invention is biologically active.

The FVII and FVIIa fusion polypeptides of the invention have an extended half-life compared to wild-type FVII polypeptides and/or wild-type FVIIa polypeptides, respectively.

“FIX activation peptide” or “FX activation peptide” refers to the peptide that is released from the mature peptide when the mature FIX or FX polypeptide, respectively, is converted from its inactive (zymogen) form to its active form.

The FIX and FX activation peptides may be truncated. The terms “truncated FIX peptide” and “truncated FX peptide” includes a FIX peptide or FX peptide, respectively, that does not comprise the first 1, 2, 3, 4, 5 or 6 amino acid residues present at the C-terminal and/or N-terminal of a wild-type or homologue FIX or FX activation peptide. A truncated FIX or FX activation peptide is biologically active.

The FX or FIX activation peptide of the invention may also be extended. The term “extended FX or FIX activation peptide” as used herein includes a FX or FIX activation peptide that comprises an additional 1, 2, 3, 4, 5, 6 or more amino acid residues at the N-terminal and/or C-terminal of the wild-type or homologous activation peptide. An extended FVII or FVIIa polypeptide is biologically active.

The sequence of a wild-type FVII polypeptide and a wild-type FVIIa polypeptide is as set forth in SEQ ID NO:1. This sequence represents a mature FVII or FVIIa polypeptide without a leader sequence.

The three-letter indication “GLA” in SEQ ID NO:1 means 4-carboxyglutamic acid (y-carboxyglutamate).

The sequence of a wild-type activation peptide of FIX is set forth in SEQ ID NO:2. The sequence of a wild-type activation peptide of FX is set forth in SEQ ID NO:3.

In one embodiment of the invention, the invention relates to a polypeptide comprising residues 1, 2, 3, 4, 5, 6, 7, 8, 9 or 10 to 397, 398, 399, 400, 401, 402, 403, 404, 405 or 406 of an amino acid sequence as set forth in SEQ ID NO:1, or a homologue thereof, and the activation peptide comprises residues 1, 2, 3, 4, 5 or 6 to 30, 21, 32, 33, 34 or 35 of an amino acid sequence as set forth in SEQ ID NO:2 or a homologue thereof or residues 1, 2, 3, 4, 5 or 6 to 47, 48, 49, 50, 51 or 52 of an amino acid sequence as set forth in SEQ ID NO:3 or a homologue thereof.

Thus, the invention includes polypeptide sequences beginning with any one of residues 1, 2, 3, 4, 5, 6, 7, 8, 9 or 10 of SEQ ID NO:1 and ending with any one of residues 397, 398, 399, 400, 401, 402, 403, 404 or 405 of SEQ ID NO:1 fused at the C-terminal to an activation peptide that begins with any one of residues 1, 2, 3, 4, 5 or 6 of SEQ ID NO:2 and ends with any one of residues 30, 31, 32, 33, 34 or 35 of SEQ ID NO:2 or begins with any one of residues 1, 2, 3, 4, 5 or 6 of SEQ ID NO:3 and ends with any one of residues 47, 48, 49, 50, 51 or 52 of SEQ ID NO:3.

The term “polypeptide” in this text means, in general terms, a plurality of amino acid residues joined together by peptide bonds. It is used interchangeably and means the same as peptide, oligopeptide, oligomer or protein, and includes glycoproteins and derivatives thereof. The term “polypeptide” is also intended to include and homologues, wherein the homologue retains the same (or higher) biological activity as a reference protein.

In the present invention a homologue of a FVII or FVIIa polypeptide has the same or higher biological activity as the wild-type FVII or FVIIa polypeptide set out in SEQ ID NO:1. In the present text, the term homologue includes variants.

The biological activity of a wild-type FVIIa is the ability of the polypeptide to convert its substrate FX to the active FXa. Where a polypeptide of the invention is a FVII polypeptide, it must first be activated to FVIIa before its biological activity can be determined.

The biological activity of a FVIIa polypeptide may be defined using an “In vitro proteolysis assay” as follows:

Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min in a microtiter plate. FX cleavage is then stopped by the addition of 50 microL 50 mM Hepes, pH 7.4, containing 0.1 M NaCl, 20 mM EDTA and 1 mg/ml bovine serum albumin. The amount of FXa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of homologue FVIIa and wild-type Factor VIIa (as set out in SEQ ID NO:1):

Ratio=(A_{405 nm} Factor VIIa homologue)/(A_{405 nm} Factor VIIa polypeptide as set out in SEQ ID NO:1).

In this test, a ratio of above 0.8 (optionally including about or above 1, 1.25 and 1.5) defines a biologically active polypeptide according to the invention.

In one embodiment, the FVII or FVIIa polypeptide of the invention extends to amino acid sequences that are at least 80% homologous with the wild-type FVII or FVIIa polypeptide defined in SEQ ID NO:1. In further embodiments, such sequences may be at least 85%, 86%, 87%, 88%, 89%, 90%, 95%, 96%, 97%, 98%, 99%, 99.4% or 99.5% homologous with/identical to the amino acid sequence of the wild-type FVII or FVIIa polypeptide in SEQ ID NO:1.

As is well understood, homology at the amino acid level is generally in terms of amino acid similarity or identity.

Percent homology of sequences may be determined by visual inspection and mathematical calculation. Alignment for purposes of determining percent amino acid sequence identity can be achieved in various ways using publicly available computer software such as BLAST or ALIGN. For example, protein searches can be performed with the XBLAST program to obtain amino acid sequences homologous to protein molecules of the invention. Those skilled in the art can determine appropriate parameters for measuring alignment, including any algorithms needed to achieve maximal alignment over the full length of the sequences being compared.

A homologue of a FVIIa polypeptide of the invention has the same or higher biological activity of the wild-type FVIIa polypeptide in SEQ ID NO:1. A homologue of a FVII polypeptide of the invention has the same or higher biological activity of the wild-type FVIIa polypeptide in SEQ ID NO:1, once the FVII polypeptide is activated to a FVIIa polypeptide. A variant polypeptide of the invention includes all variant polypeptide sequences disclosed in US2006/0166915, WO02/077218, US2003/0100075, US2003/0130191 and WO2004/029090 and these disclosures are fully incorporated herein by reference.

In one embodiment, the FIX or FX activation peptide of the invention extends to amino acid sequences that are at least 80% homologous with the wild-type FIX or FX activation peptide as in SEQ ID NO:2 or NO: 3, respectively. In further embodiments, such sequences may be at least 85%, 86%, 87%, 88%, 89%, 90%, 95%, 96%, 97%, 98%, 99%, 99.4% or 99.5% homologous with/identical to the amino acid sequence of such wild-type FIX or FX activation peptide.

The biological activity of the FIX or FX activation peptide in the context of the present invention is the ability to increase the plasma half-life of a fusion polypeptide of the invention above the plasma half-life of the FVII or FVIIa polypeptide before the activation peptide is added. The half-life of the fusion polypeptide can be about or above 5%, 10%, 15%, 20%, 30%, 40%, 50%, 100% or 200% more than the half-life of the FVII or FVIIa polypeptide before the activation peptide is added.

The term plasma “half-life” means the time it takes until only 50% of the initial protein pool for a particular protein remains. Half-life can be measured by ELISA.

A homologue of a FVII or FVIIa polypeptide or a FIX or FX peptide of the invention may be (i) one in which one or more of the amino acid residues are substituted with a conserved or non-conserved amino acid residue (preferably, a conserved amino acid residue) and such substituted amino acid residue may or may not be one encoded by the genetic code or (ii) one in which one or more of the amino acid residues includes a substituent group.

Preferred are homologues having the amino acid sequence of the polypeptides defined above in which several e.g. 5 to 10, 1 to 5, 1 to 3, 2, 1 or no amino acid residues are substituted, deleted or added in any combination. Especially preferred among these are silent substitutions, additions and deletions, which do not alter the properties and activities of the protein of the present invention. Also especially preferred in this regard are conservative substitutions.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi-conservative” amino acid substitutions.

Amino acid deletions may also be made relative to the amino acid sequence for the fusion polypeptide referred to above. Thus, for example, amino acids which do not have a substantial effect on the activity of the polypeptide, or at least which do not eliminate such activity, may be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

Amino acid insertions relative to the sequence of the fusion polypeptide above can also be made. This may be done to alter the properties of a substance of the present invention (e.g. to assist in identification, purification or expression, as explained above in relation to fusion polypeptides).

Amino acid changes relative to the sequence given in a) above can be made using any suitable technique e.g. by using site-directed mutagenesis.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

In a further embodiment of the invention, the FVII or FVIIa polypeptide of the invention extends to homologues comprising one or more substitutions relative to the amino acid sequence of SEQ ID NO:1, wherein said substitutions are replacements with any one or more amino acids at a position selected from 172, 173, 175, 176, 177, 196, 197, 198, 199, 200, 203, 235, 237, 238, 239, 240, 286, 287, 288, 289, 290, 291, 292, 293, 294, 295, 297, 299, 319, 320, 321, 327, 341, 363, 364, 365, 366, 367, 370 or 373 corresponding to amino acid positions of SEQ ID NO:1.

The term “any one or more amino acid” as used herein means one or more amino acid that are different from the amino acid in SEQ ID NO:1. This includes but is not limited to amino acids that can be encoded by a polynucleotide. In one embodiment the different amino acid is in natural L-form and can be encoded by a polynucleotide, a specific example being L-cysteine (Cys).

In the following embodiments, the expression “at least” means either that only the amino acid specified is substituted or that the specific amino acid substituted is in addition to one or more other amino acid changes in the molecule.

In the following embodiments, the amino acid substitutions are in reference to SEQ ID NO:1 as follows: the letter represents the amino acid naturally present at a position in SEQ ID NO:1 and the following number represents the position in SEQ ID NO:1.

In a further embodiment of the invention, the Factor VII or VIIa polypeptide is a homologue wherein at least G237 is replaced with any one or more amino acid selected from Ala, Val, Leu, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least T238 is replaced with any one or more amino acid selected from Ala, Gly, Val, Leu, Ile, Phe, Met, Trp, Tyr, Asp, Asn, Glu, Gln, His, Lys, Arg, Cys or Ser.

In a further embodiment of the invention, the Factor VII or VIIa polypeptide is a homologue wherein at least T239 is replaced with any one or more amino acid selected from Gly, Ala, Val, Leu, Ile, Phe, Met, Trp, Tyr, Asp, Asn, Glu, Gln, His, Lys, Arg, Cys or Ser.

In a further embodiment of the invention, the Factor VII or VIIa polypeptide is a homologue wherein at least Q286 is replaced with any one or more amino acid selected from Gly, Ala, Val, Leu, Ile, Phe, Met, Trp, Tyr, Asp, Asn, Glu, His, Lys, Arg, Cys, Thr or Ser.

In a further embodiment of the invention, the Factor VII or VIIa polypeptide is a homologue wherein at least L287 is replaced with any one or more amino acid selected from Gly, Ala, Val, Ile, Phe, Met, Tip, Tyr, Asp, Asn, Glu, Gln, His, Lys, Arg, Cys, Thr or Ser.

In a further embodiment of the invention, the Factor VII or VIIa polypeptide is a homologue wherein at least L288 is replaced with any one or more amino acid selected from Gly, Ala, Val, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least D289 is replaced with any one or more amino acid selected from Gly, Ala, Val, Leu, Ile, Phe, Met, Trp, Tyr, Asn, Glu, Gln, His, Lys, Arg, Cys, Ser or Thr.

In a further embodiment of the invention, the Factor VII or VIIa polypeptide is a polypeptide wherein R290 is replaced with any one or more amino acid selected from Gly, Ala, Val, Leu, Ile, Phe, Met, Trp, Tyr, Asp, Asn, Glu, Gln, His, Lys, Cys, Thr or Ser.

In a further embodiment of the invention, the Factor VII or VIIa polypeptide is a homologue wherein at least A292 is replaced with any one or more amino acid selected from Gly, Val, Leu, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least T293 is replaced with any one or more amino acid selected from Gly, Ala, Val, Leu, Ile, Phe, Met, Trp, Tyr, Asp, Asn, Glu, Gln, His, Lys, Arg, Cys or Ser.

In a further embodiment of the invention, the Factor VII or VIIa polypeptide is a homologue wherein at least A294 is replaced with any one or more amino acid selected from Gly, Val, Leu, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least L295 is replaced with any one or more amino acid selected from Gly, Ala, Val, Ile, Phe, Met, Tip, Tyr, Asp, Asn, Glu, Gln, His, Lys, Arg, Cys, Thr or Ser.

In a further embodiment of the invention, the Factor VII or VIIa polypeptide is a homologue wherein at least L297 is replaced with any one or more amino acid selected from Gly, Ala, Val, Ile, Phe, Met, Tip, Tyr, Asp, Asn, Glu, Gln, His, Lys, Arg, Cys, Thr or Ser.

In a further embodiment of the invention, the Factor VII or VIIa polypeptide is a homologue wherein at least V299 is replaced with any one or more amino acid selected from Gly, Ala, Leu, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least M327 is replaced with any one or more amino acid selected from Gly, Ala, Val, Leu, Ile, Phe, Trp, Tyr, Asp, Asn, Glu, Gln, His, Lys, Arg, Cys, Ser or Thr.

In a further embodiment of the invention, the Factor VII or VIIa polypeptide is a homologue wherein at least K341 is replaced with any one or more amino acid selected from Gly, Ala, Val, Leu, Ile, Phe, Met, Trp, Tyr, Asp, Asn, Glu, Gln, His, Arg, Cys, Ser or Thr.

In a further embodiment of the invention, the Factor VII or VIIa polypeptide is a homologue wherein at least S363 is replaced with any one or more amino acid selected from Gly, Ala, Val, Leu, Ile, Phe, Met, Trp, Tyr, Asp, Asn, Glu, Gln, His, Lys, Arg, Cys or Thr.

In a further embodiment of the invention, the Factor VII or VIIa polypeptide is a homologue wherein at least W364 is replaced with any one or more amino acid selected from Gly, Ala, Val, Leu, Ile, Phe, Met, Tyr, Asp, Asn, Glu, Gln, His, Lys, Arg, Cys, Thr or Ser.

In a further embodiment of the invention, the Factor VII or VIIa polypeptide is a homologue wherein at least G365 is replaced with any one or more amino acid selected from Ala, Val, Leu, Ile, Phe, Met, Trp, Tyr, Asp, Asn, Glu, Gln, His, Lys, Arg, Cys, Thr or Ser.

In a further embodiment of the invention, the Factor VII or VIIa polypeptide is a homologue wherein at least Q366 is replaced with any one or more amino acid selected from Gly, Ala, Val, Leu, Ile, Phe, Met, Trp, Tyr, Asp, Asn, Glu, His, Lys, Arg, Cys, Thr or Ser.

In a further embodiment of the invention, the Factor VII or VIIa polypeptide is a homologue wherein at least Q366 is replaced with any one or more amino acid selected from Val, Leu, Ile, Phe, Met, Tip, Tyr, Asp, Asn, His, Lys, Arg, Cys, Thr or Ser.

In a further embodiment of the invention, the Factor VII or VIIa polypeptide is a homologue wherein at least V172 is replaced with any one or more amino acid selected from Gly, Ala, Leu, Ile, Phe, Met, Trp, Tyr, Asp, Asn, Glu, Gln, His, Lys, Arg, Cys, Ser or Thr.

In a further embodiment of the invention, the Factor VII or VIIa polypeptide is a homologue wherein at least N173 is replaced with any one or more amino acid selected from Gly, Ala, Val, Leu, Ile, Phe, Met, Trp, Tyr, Asp, Glu, Gln, His, Lys, Arg, Cys, Ser or Thr.

In a further embodiment of the invention, the Factor VII or VIIa polypeptide is a homologue wherein at least A175 is replaced with any one or more amino acid selected from Gly, Leu, Ile, Phe, Met, Trp, Tyr, Asp, Asn, Glu, Gln, His, Lys, Arg, Cys, Ser, Val or Thr.

In a further embodiment of the invention, the Factor VII or VIIa polypeptide is a homologue wherein at least Q176 is replaced with any one or more amino acid selected from Gly, Ala, Leu, Ile, Phe, Met, Trp, Tyr, Asp, Asn, Glu, His, Lys, Arg, Cys, Ser, Val or Thr.

In a further embodiment of the invention, the Factor VII or VIIa polypeptide is a homologue wherein at least L177 is replaced with any one or more amino acid selected from Gly, Ala, Ile, Phe, Met, Trp, Tyr, Asp, Asn, Glu, Gln, His, Lys, Arg, Cys, Ser, Val or Thr.

In a further embodiment of the invention, the Factor VII or VIIa polypeptide is a homologue wherein at least D196 is replaced with any one or more amino acid selected from Gly, Ala, Leu, Ile, Phe, Met, Trp, Tyr, Asn, Glu, Gln, His, Lys, Arg, Cys, Ser, Val or Thr.

In a further embodiment of the invention, the Factor VII or VIIa polypeptide is a homologue wherein at least K197 is replaced with any one or more amino acid selected from Gly, Ala, Leu, Ile, Phe, Met, Trp, Tyr, Asp, Asn, Glu, Gln, His, Arg, Cys, Ser, Val or Thr.

In a further embodiment of the invention, the Factor VII or VIIa polypeptide is a homologue wherein at least I198 is replaced with any one or more amino acid selected from Gly, Ala, Leu, Phe, Met, Tip, Tyr, Asp, Asn, Glu, Gln, His, Lys, Arg, Cys, Ser, Val or Thr.

In a further embodiment of the invention, the Factor VII or VIIa polypeptide is a homologue wherein at least K199 is replaced with any one or more amino acid selected from Gly, Ala, Leu, Ile, Phe, Met, Trp, Tyr, Asp, Asn, Glu, Gln, His, Arg, Cys, Ser, Val or Thr.

In a further embodiment of the invention, the Factor VII or VIIa polypeptide is a homologue wherein at least N200 is replaced with any one or more amino acid selected from Gly, Ala, Val, Leu, Ile, Phe, Met, Trp, Tyr, Asp, Glu, Gln, His, Lys, Arg, Cys, Ser or Thr.

In a further embodiment of the invention, the Factor VII or VIIa polypeptide is a homologue wherein at least N203 is replaced with any one or more amino acid selected from Gly, Ala, Val, Leu, Ile, Phe, Met, Trp, Tyr, Asp, Glu, Gln, His, Lys, Arg, Cys, Ser or Thr.

In a further embodiment of the invention, the Factor VII or VIIa polypeptide is a homologue wherein at least V235 is replaced with any one or more amino acid selected from Gly, Ala, Leu, Ile, Phe, Met, Trp, Tyr, Asp, Asn, Glu, Gln, His, Lys, Arg, Cys, Ser or Thr.

In a further embodiment of the invention, the Factor VII or VIIa polypeptide is a homologue wherein at least N240 is replaced with any one or more amino acid selected from Gly, Ala, Val, Leu, Ile, Phe, Met, Trp, Tyr, Asp, Glu, Gln, His, Lys, Arg, Cys, Ser or Thr.

In a further embodiment of the invention, the Factor VII or VIIa polypeptide is a homologue wherein at least D319 is replaced with any one or more amino acid selected from Gly, Ala, Val, Leu, Ile, Phe, Met, Trp, Tyr, Asn, Glu, Gln, His, Lys, Arg, Cys, Ser or Thr.

In a further embodiment of the invention, the Factor VII or VIIa polypeptide is a homologue wherein at least S320 is replaced with any one or more amino acid selected from Gly, Ala, Val, Leu, Ile, Phe, Met, Trp, Tyr, Asp, Asn, Glu, Gln, His, Lys, Arg, Cys or Thr.

In a further embodiment of the invention, the Factor VII or VIIa polypeptide is a homologue wherein at least P321 is replaced with any one or more amino acid selected from Gly, Ala, Val, Leu, Ile, Phe, Met, Trp, Tyr, Asp, Asn, Glu, Gln, His, Lys, Arg, Cys, Ser or Thr.

In a further embodiment of the invention, the Factor VII or VIIa polypeptide is a homologue wherein at least G367 is replaced with any one or more amino acid selected from Ala, Val, Leu, Ile, Phe, Met, Trp, Tyr, Asp, Asn, Glu, Gln, His, Lys, Arg, Cys, Ser or Thr.

In a further embodiment of the invention, the Factor VII or VIIa polypeptide is a homologue wherein at least T370 is replaced with any one or more amino acid selected from Gly, Ala, Val, Leu, Ile, Phe, Met, Trp, Tyr, Asp, Asn, Glu, Gln, His, Lys, Arg, Cys or Ser.

In a further embodiment of the invention, the Factor VII or VIIa polypeptide is a homologue wherein at least H373 is replaced with any one or more amino acid selected from Gly, Ala, Val, Leu, Ile, Phe, Met, Trp, Tyr, Asp, Asn, Glu, Gln, Lys, Arg, Cys, Ser or Thr.

In a further embodiment of the invention, the Factor VII or VIIa polypeptide is a homologue wherein at least i) L287 is replaced with an amino acid selected from Thr or Ser and ii) A294 is replaced with an amino acid selected from Thr or Ser, and iii) M298 has been replaced by a Lys.

In a further embodiment of the invention, the Factor VII or VIIa polypeptide is a homologue wherein at least i) L287 is replaced with a amino acid selected from Thr or Ser and ii) A294 is replaced with an amino acid selected from Thr or Ser, and iii) M298 has been replaced by a Lys, and iv) E296 has been replaced by an amino acid selected from the group consisting of Ile, Leu, Thr or Val.

In a further embodiment of the invention, the Factor VII or VIIa polypeptide is a homologue wherein at least i) L287 is replaced with an amino acid selected from Thr or Ser and ii) A294 is replaced with an amino acid selected from Thr or Ser, and iii) M298 has been replaced by a Lys, and iv) V158 has been replaced by an amino acid selected from the group consisting of Asp or Glu.

In a further embodiment of the invention, the Factor VII or VIIa polypeptide is a homologue wherein at least i) L287 is replaced with an amino acid selected from Thr or Ser and ii) A294 is replaced with an amino acid selected from Thr or Ser, and iii) M298 has been replaced by a Lys, and iv) E296 has been replaced by an amino acid selected from the group consisting of Ile, Leu, Thr or Val, and v) V158 has been replaced by an amino acid selected from the group consisting of Asp or Glu.

The terminology for amino acid substitutions described below in reference to SEQ ID NO:1 is as follows:

The first letter represents the amino acid naturally present at a position of SEQ ID NO:1. The following number represents the position in SEQ ID NO:1. The second letter represents the different amino acid substituting for the natural amino acid. An example is K197A-FVII, wherein the Lysine at position 197 of SEQ ID NO:1 is replaced by a Alanine.

In a further embodiment of the invention, the Factor VII or VIIa polypeptide is a polypeptide selected from the group consisting of:

V158D/L287T/A294S/E2961/M298K-FVIIA; V158D/L287T/A294S/E296V/M298K-FVIIA; V158D/L287T/A294S/E296L/M298K-FVIIA; V158D/L287T/A294S/E296T/M298K-FVIIA; V158D/L287T/A294T/E2961/M298K-FVIIA; V158D/L287T/A294T/E296V/M298K-FVIIA; V158D/L287T/A294T/E296L/M298K-FVIIA; V158D/L287T/A294T/E296T/M298K-FVIIA; V158D/L287S/A294S/E2961/M298K-FVIIA; V158D/L287S/A294S/E296V/M298K-FVIIA; V158D/L287S/A294S/E296L/M298K-FVIIA; V158D/L287S/A294S/E296T/M298K-FVIIA; V158D/L287S/A294T/E2961/M298K-FVIIA; V158D/L287S/A294T/E296V/M298K-FVIIA; V158D/L287S/A294T/E296L/M298K-FVIIA; V158D/L287S/A294T/E296T/M298K-FVIIA; V158E/L287T/A294S/E2961/M298K-FVIIA; V158E/L287T/A294S/E296V/M298K-FVIIA; V158E/L287T/A294S/E296L/M298K-FVIIA; V158E/L287T/A294S/E296T/M298K-FVIIA; V158E/L287T/A294T/E2961/M298K-FVIIA; V158E/L287T/A294T/E296V/M298K-FVIIA; V158E/L287T/A294T/E296L/M298K-FVIIA; V158E/L287T/A294T/E296T/M298K-FVIIA; V158E/L287S/A294S/E2961/M298K-FVIIA; V158E/L287S/A294S/E296V/M298K-FVIIA; V158E/L287S/A294S/E296L/M298K-FVIIA; V158E/L287S/A294S/E296T/M298K-FVIIA; V158E/L287S/A294T/E2961/M298K-FVIIA; V158E/L287S/A294T/E296V/M298K-FVIIA; V158E/L287S/A294T/E296L/M298K-FVIIA, V158E/L287S/A294T/E296T/M298K-FVIIA, Q176A-FVII, Q176L-FVII L177S-FVII, D196A-FVII, K197A-FVII, K199A-FVII, T238A-FVII, T239I-FVII, T239Y-FVII, Q286A-FVII, D289E-FVII, D289R-FVII, R290Q-FVII, M327Q-FVII, M327N-FVII, K341A-FVII, K341E-FVII, K341Q-FVII, S363M-FVII, S363A-FVII, W364H-FVII or Q366E-FVII.

Examples of further substitutions in the FVIIa polypeptides of the present invention include, without limitation, L305V, L305V/M306D/D309S, L3051, L305T, F374P, F374Y, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298N, M298N, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, M298Q/K337A, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A, L305V/V158D/E296V/M298Q/K337A, L305V/V158T/E296V/M298Q/K337A, L305V/S314E/F374Y, L305V/K337A, L305V/K337A/F374Y, L305V/S314E/K337A/F374Y, S314E, V317N, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316QN158D, K316Q/E296V, K316Q/M298Q, K316QN158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337A/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, D212N, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298QN158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E, S52A, S60A; R152E, S344A, T106N, K143N/N145T, V253N, R290N/A292T, G291N, R315N/V317T, K143N/N145T/R315N/V317T; and substitutions, additions or deletions in the amino acid sequence from 233Thr to 240Asn; substitutions, additions or deletions in the amino acid sequence from 304Arg to 329Cys; and substitutions, additions or deletions in the amino acid sequence from 153Ile to 223Arg.

In a further embodiment of the invention, the Factor VII or VIIa polypeptide is a homologue wherein at least one amino acid in the remaining positions in the protease domain has been replaced with any other amino acid. “Remaining positions” means any position in the protease domain of SEQ ID NO:1 where the amino acid has not yet been substituted for a different amino acid.

In a further embodiment of the invention, the Factor VII or VIIa polypeptide is a homologue wherein at most 20 additional amino acids in the remaining positions in the protease domain have been replaced with any other amino acids.

In a further embodiment of the invention, the Factor VII or VIIa polypeptide is a homologue wherein at least one amino acid corresponding to an amino acid at a position selected from 157-170 of SEQ ID NO:1 has been replaced with any other amino acid.

In a further embodiment of the invention, the Factor VII or VIIa polypeptide is a homologue wherein at least one amino acid corresponding to an amino acid at a position selected from 290-305 of SEQ ID NO:1 has been replaced with any other amino acid.

In a further embodiment of the invention, the Factor VII or VIIa polypeptide is a homologue wherein at least R304 has been replaced by an amino acid selected from the group consisting of Tyr, Phe, Leu or Met.

In a further embodiment of the invention, the Factor VII or VIIa polypeptide is a homologue wherein at least one amino acid corresponding to an amino acid at a position selected from 306-312 of SEQ ID NO:1 has been replaced with any other amino acid.

In a further embodiment of the invention, the Factor VII or VIIa polypeptide is a homologue wherein at least M306 has been replaced by an amino acid selected from the group consisting of Asp or Asn.

In a further embodiment of the invention, the Factor VII or VIIa polypeptide is a homologue wherein at least D309 has been replaced by an amino acid selected from the group consisting of Ser or Thr.

In a further embodiment of the invention, the Factor VII or VIIa polypeptide is a homologue wherein at least one amino acid corresponding to an amino acid at a position selected from 330-339 of SEQ ID NO:1 has been replaced with any other amino acid.

In a further embodiment of the invention, the Factor VII or VIIa polypeptide is a homologue wherein at least A274 has been replaced with any other amino acid.

In a further embodiment of the invention, the Factor VII or VIIa polypeptide is a homologue wherein at least A274 has been replaced by an amino acid selected from the group consisting of Met, Leu, Lys or Arg.

In a further embodiment of the invention, the Factor VII or VIIa polypeptide is a homologue wherein at least K157 has been replaced by an amino acid selected from the group consisting of Gly, Val, Ser, Thr, Asn, Gln, Asp or Glu.

In a further embodiment of the invention, the Factor VII or VIIa polypeptide is a homologue wherein at least K337 has been replaced by an amino acid selected from the group consisting of Ala, Gly, Val, Ser, Thr, Asn, Gln, Asp or Glu.

In a further embodiment of the invention, the Factor VII or VIIa polypeptide is a homologue wherein at least D334 has been replaced by an amino acid selected from the group consisting of Gly or Glu.

In a further embodiment of the invention, the Factor VII or VIIa polypeptide is a homologue wherein at least S336 has been replaced by an amino acid selected from the group consisting of Gly or Glu.

In a further embodiment of the invention, the Factor VII or VIIa polypeptide is a homologue wherein at least V158 has been replaced by an amino acid selected from the group consisting of Ser, Thr, Asn, Gln, Asp or Glu.

In a further embodiment of the invention, the Factor VII or VIIa polypeptide is a homologue wherein at least E296 has been replaced by an amino acid selected from the group consisting of Arg, Lys, Ile, Leu, Thr or Val.

In a further embodiment of the invention, the Factor VII or VIIa polypeptide is a homologue wherein at least M298 has been replaced by an amino acid selected from the group consisting of Lys, Arg, Gln or Asn.

In a further embodiment of the invention, the Factor VII or VIIa polypeptide is a homologue wherein at least L305 has been replaced by an amino acid selected from the group consisting of Val, Tyr or Ile.

In a further embodiment of the invention, the Factor VII or VIIa polypeptide is a homologue wherein at least S314 has been replaced by an amino acid selected from the group consisting of Gly, Lys, Gln or Glu.

In a further embodiment of the invention, the Factor VII or VIIa polypeptide is a homologue wherein at least F374 has been replaced by an amino acid selected from the group consisting of Pro or Tyr.

In a further embodiment of the invention, the homologue further comprises replacement of a one or more amino acids in the N-terminal Gla domain (amino acids at position corresponding to 1-37 of SEQ ID NO:1), thereby providing Factor VIIa polypeptides with a substantially higher affinity for membrane phospholipids, such as membrane phospholipids of tissue factor-bearing cells or of platelets, thereby generating Factor VII polypeptide homologues which have an improved procoagulant effect. Thus, the Factor VIIa polypeptide homologues mentioned directly above may, in addition to any optional amino acid changes, also have at least one amino acid replaced in the N-terminal GLA domain. In one embodiment, one or more amino acids in positions selected from 4, 8, 10, 11, 28, 32, 33, 34 or 35 (referring to SEQ ID NO:1) is replaced with a different amino acid. In one embodiment, one or more amino acids in positions selected from positions 10 or 32 (referring to SEQ ID NO:1) is replaced with a different amino acid. Examples of preferred amino acids to be incorporated in the above-mentioned positions are: The amino acid Pro in position 10 is replaced by Gln, Arg, His, Gln, Asn or Lys; and/or the amino acid Lys in position 32 is replaced by Glu, Gln or Asn.

Other amino acids in the GLA domain, based on the different phospholipid affinities and sequences of the vitamin K-dependent plasma proteins, may also be substituted.

The term “N-terminal GLA-domain” means the amino acid sequence 1-37 of Factor VII.

The three-letter indication “GLA” means 4-carboxyglutamic acid (y-carboxyglutamate).

The term “protease domain” means the amino acid sequence 153-406 of Factor VII (the heavy-chain of Factor VIIa).

The polypeptides of the invention can comprise post-translational modifications, in particular the following modifications: 10 gamma-carboxylated, N-terminally located glutamic acid residues, 1 beta-hydroxylated aspartic acid residue, and 2 N-glycosylated asparagine residues.

In one embodiment, the polypeptides of the invention are isolated.

The term “isolated” as used herein to describe the various polypeptides disclosed herein, means polypeptide that has been identified and separated and/or recovered from a component of its natural environment.

In a further embodiment, a polypeptide of the invention is a recombinant polypeptide.

The term “fusion polypeptide” in this text means, in general terms, one or more polypeptides sequences joined together by chemical means, or by peptide bonds through protein synthesis or both.

The second aspect of the invention relates to a nucleic acid sequence, for example a RNA sequence or a DNA sequence, encoding a polypeptide of the invention.

The nucleic acid sequences of the invention are useful in the production of polypeptides of the invention.

The nucleic acid sequence of the invention can additionally comprise a promoter or other regulatory sequence which controls expression of the nucleic acid. Promoters and other regulatory sequences which control expression of a nucleic acid have been identified and are known in the art. It may not be necessary to utilise the whole promoter or other regulatory sequence. Only the minimum essential regulatory element may be required and, in fact, such elements can be used to construct chimeric sequences or other promoters. The essential requirement is, of course, to retain the tissue and/or temporal specificity. The promoter may be any suitable known promoter, for example, the human cytomegalovirus (CMV) promoter, the CMV immediate early promoter, the HSV thymidinekinase, the early and late SV40 promoters or the promoters of retroviral LTRs, such as those of the Rous Sarcoma virus (RSV) and metallothionine promoters such as the mouse metallothionine-I promoter. The promoter may comprise the minimum comprised for promoter activity (such as a TATA elements without enhancer elements) for example, the minimum sequence of the CMV promoter.

Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence.

The polymerase chain reaction (PCR) procedure may be employed to isolate and amplify a DNA sequence encoding a desired protein sequence. Oligonucleotides that define the desired termini of the DNA sequence are employed as 5′ and 3′ primers. The oligonucleotides may additionally contain recognition sites for restriction endonucleases, to facilitate insertion of the amplified DNA sequence into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et al., eds., Academic Press, Inc. (1990).

The third aspect of the invention relates to a vector comprising the nucleic acid sequence of the second aspect of the invention.

The present invention also provides recombinant cloning and expression vectors containing DNA encoding the polypeptides of the invention

The fourth aspect of the invention relates to a host cell comprising the nucleic acid sequence of the second aspect of the invention or the vector of the third aspect of the invention.

Vectors and host cells comprising nucleic acid sequences of the invention may be used to prepare the polypeptides of the invention encoded by the nucleic acid sequences.

Vectors of the invention may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host, may be used for expression in this regard.

The fifth aspect of the invention extends to a method of producing a FVII fusion polypeptide comprising culturing the host cell of the fourth aspect of the invention under conditions that promote expression of the polypeptide and recovering the expressed polypeptide from the culture.

In a further embodiment, the method of producing a FVII fusion polypeptide according to the invention further includes the step of purifying the expressed polypeptide. In a yet further embodiment, the step of purification includes cleaving a peptide bond between residue 152 and residue 153 of the polypeptide forming two polypeptide chains held together by a disulphide bridge i.e. activating the polypeptide to form a FVIIa polypeptide. This step can be achieved by using one or more proteases to proteolytically convert a FVII polypeptide to the active two-chain form.

In a preferred embodiment, recombinant FVII is secreted into culture media in its single-chain form and is then proteolytically converted by autocatalysis to the active two-chain form, FVIIa, during a chromatographic purification process.

FVII and FVIIa can also be purified from naturally occurring cells and fused to the FIX or FX activation peptide sequences.

Expression, isolation and purification of the polypeptides of the invention may be accomplished by any suitable technique, including but not limited to the following:

Any suitable expression system may be employed. An origin of replication that confers the ability to replicate in the desired host cells, and a selection gene by which transformants are identified, may be incorporated into an expression vector used to produce a polypeptide of the invention. In addition, a sequence encoding an appropriate signal peptide (native or heterologous) can be incorporated into expression vectors. For example, a sequence encoding the pre-pro leader sequence of FVII, FIX, prothrombin, protein C or protein S can be incorporated. A DNA sequence for a signal peptide (secretory leader) may be fused in frame to the nucleic acid sequence of the invention so that the DNA is initially transcribed and the mRNA translated into a fusion polypeptides comprising the signal peptide. A signal peptide that is functional in the intended host cells promotes extracellular secretion of the polypeptide. The signal peptide is cleaved from the polypeptide during translation, but allows secretion of polypeptide from the cell.

Suitable host cells for expression of polypeptides of the invention include any cell that is capable of producing posttranslationally polypeptides and includes yeast, fungi, insect and higher eukaryotic cells. Mammalian cells, and particularly human embryonic kidney (HEK), Chinese hamster ovary (CHO) or baby hamster kidney (BHK) cells, are particularly preferred for use as host cells. Appropriate cloning and expression vectors for use with mammalian and yeast hosts are described, for example, in Pouwels et al. Cloning Vectors: A Laboratory Manual, Elsevier, N.Y., (1986) (ISBN 0444904018).

Established cell lines of mammalian origin also may be employed, for example, CHO (e.g., ATCC CCL 61), COS-1 (e.g., ATCC CRL 1650) and HEK293 (e.g., ATCC CRL 1573) cell lines. In a preferred embodiment, the HEK-293F cell line is used.

Established methods for introducing DNA into mammalian cells have been described (Kaufman, R. J., Large Scale Mammalian Cell Culture, 1990, pp. 15-69). Additional protocols using commercially available reagents, such as Lipofectamine or Lipofectamine 2000 lipid reagents (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. Other examples of selectable markers that can be incorporated into an expression vector include cDNAs conferring resistance to antibiotics, such as G418 (Geneticin) and hygromycin B. Cells harbouring the vector can be selected on the basis of resistance to these compounds.

Additional control sequences shown to improve expression of heterologous genes from mammalian expression vectors can be used. For example, the expression augmenting sequence element (EASE) derived from CHO cells (Morris et al., Animal Cell Technology, 1997, pp. 529-534).

Yeast host cells, preferably from the Saccharomyces genus (e.g., S. cerevisiae), can also be used. Other genera of yeast, such as Pichia (Pichia pastoris) or Kluyveromyces, may also be employed. Yeast vectors will often contain an origin of replication sequence from a 2 [mu] yeast plasmid, an autonomously replicating sequence (ARS), a promoter region, sequences for polyadenylation, sequences for transcription termination, and a selectable marker gene.

Suitable promoter sequences for yeast vectors include, among others, promoters for metallothionine, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase.

The yeast [alpha]-factor leader sequence may be employed to direct secretion of the polypeptide. The [alpha]-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978.

A FVII and/or FVIIa polypeptide according to the invention can be prepared using animal or plant transgenic technology.

For example, the polypeptides of the invention can be produced within the mammary glands of a host female mammal. This is further discussed in US2006/0166915, the contents of which is incorporated herein.

Production in transgenic plants may also be employed. Expression may be generalised or directed to a particular organ, such as a tuber (see, Hiatt, Nature 344:469-479 (1990), for example).

With respect to any type of host cell, as is known to the skilled artisan, procedures for purifying a recombinant polypeptide will vary according to such factors as the type of host cells employed and whether or not the recombinant polypeptide is secreted into the culture medium.

In general, the recombinant polypeptide can be isolated from the host cells if not secreted, or from the medium or supernatant if soluble and secreted, followed by one or more concentration, salting-out, ion exchange, hydrophobic interaction, affinity purification or size exclusion chromatography steps.

As to specific ways to accomplish these steps, the culture medium first can be concentrated using a commercially available protein concentration filter, for example, a Millipore Pellicon ultrafiltration unit. Following the concentration step, the concentrate can be applied to a purification matrix such as a gel filtration medium.

Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. In addition, a chromatofocusing step can be employed. Alternatively, a hydrophobic interaction chromatography step can be employed. Suitable matrices can be phenyl or octyl moieties bound to resins. In addition, affinity chromatography with a matrix which selectively binds the recombinant protein can be employed. Examples of such resins employed are lectin columns, dye columns, and metal-chelating columns. Finally, one or more reversed-phase high performance liquid chromatography (RP-HPLC) steps employing apolar RP-HPLC media, (e.g., silica gel or polymer resin having pendant methyl, octyl, octyldecyl or other aliphatic groups) can be employed to further purify the polypeptides. Some or all of the foregoing purification steps, in various combinations, are well known and can be employed to provide an isolated and purified recombinant protein.

Transformed mammalian host cells are preferably employed to express variant VII polypeptide as a secreted polypeptide in order to simplify purification. Secreted recombinant polypeptide from mammalian host cell fermentation can be purified by methods which are well known to those skilled in the art.

It is also possible to utilise an affinity column comprising a FVII polypeptide-binding protein, such as a monoclonal antibody generated against FVII polypeptides, for example FlA2 antibodies, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilised, or be competitively removed using the naturally occurring substrate of the affinity moiety, such as a polypeptide derived from the invention.

FVII and FVIIa fusion polypeptides of the invention can be substantially purified (to substantial homogeneity), as indicated by a single protein band upon analysis by SDS-polyacrylamide gel electrophoresis (SDS-PAGE). Purified to substantial purity means purified to more than 90% homogenous, including over 95% homogenous. The protein band may be visualised by silver staining, Coomassie blue staining, or (if the protein is radiolabelled) by autoradiography.

The skilled man will recognise that the procedure for purifying the expressed polypeptides will vary according to such factors as the type of host cells employed, and whether the polypeptide is intracellular, membrane-bound or a soluble form that is secreted from the host cell.

The sixth aspect of the invention relates to a pharmaceutical composition comprising a FVII polypeptide and/or a FVIIa polypeptide of the invention.

A pharmaceutical composition according to the invention can comprise a fusion FVII polypeptide according to the invention. In this embodiment, the FVII is activated to FVIIa after administration i.e. in the body, in accordance with processes normally responsible for activation of FVII to FVIIa, for example, FXa, FXIIa, FIXa, FVIIa, FVII-activating protease (FSAP) and thrombin. In addition or alternatively, a pharmaceutical composition according to the invention can comprise a fusion FVIIa polypeptide according to the invention and therefore activation has already occurred before the polypeptide is administered to a subject.

Pharmaceutical compositions for use in accordance with the present invention may comprise, in addition to an active ingredient (i.e. FVII or FVIIa), a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration. In a preferred embodiment, the composition is an injectable composition.

The formulation may be a liquid, for example, a physiologic salt solution containing non-phosphate buffer at pH 6.8-7.6, or a lyophilised or freeze dried powder.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilisers, buffers, antioxidants and/or other additives may be included, as required. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be in the form of microspheres, liposomes, other microparticulate delivery systems or sustained release formulations for administration to certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982).

The composition/polypeptide is preferably administered to an individual in a “therapeutically effective amount”, this being sufficient to show benefit to the individual.

The seventh aspect of the invention relates to a FVII and/or a FVIIa polypeptide of the invention for use in medicine. The use in medicine includes any bleeding disorder, which is reflected as any defect, congenital, acquired or induced, of cellular or molecular origin, that is manifested in bleedings. In one embodiment, the FVII and/or the FVIIa polypeptide of the invention is for use in treating a blood clotting deficiency, such as haemophilia A, haemophilia B, a FXI deficiency or a FVII deficiency, as well as for treating excessive or unwanted bleeding including bleeding in surgery or other tissue damage, or bleeding due to a defective platelet function, thrombocytopenia or von Willebrand's disease.

The eighth aspect of the invention relates to use of a FVII and/or a FVIIa polypeptide of the invention for manufacturing a medicament for use in treating a blood clotting deficiency or for treating excessive or unwanted bleeding, all as described above in relation to the seventh aspect of the invention.

The ninth aspect of the invention relates to a method of treating a condition associated with a blood clotting deficiency, or for treating excessive or unwanted bleeding, all as described above in relation to the seventh aspect of the invention, comprising administering a FVII and/or a FVIIa polypeptide of the invention to a subject.

The term ‘treatment’ is used herein to refer to any regimen that can benefit a human or non-human animal. In one embodiment, the human or non-human animal is in need of such treatment.

More specifically, treatment includes “therapeutic” and “prophylactic” and these types of treatment are to be considered in their broadest context. Accordingly, therapeutic and prophylactic treatment includes amelioration of the symptoms of a particular condition or preventing or otherwise reducing the risk of developing a particular condition. The term “prophylactic” may be considered as reducing the severity of or preventing the onset of a particular condition. “Prophylactic” also includes preventing reoccurrence of a particular condition in a patient previously diagnosed with the condition. “Prophylactic” does not necessarily mean that the subject will not eventually contract a disease condition.

“Therapeutic” may also reduce the severity of an existing condition and does not necessarily imply that a subject is treated until total recovery.

The FVII and FVIIa products of the invention may be administered alone but will preferably be administered as part of a pharmaceutical composition according to the sixth aspect of the invention.

The FVII and FVIIa products of the invention may be administered to a patient in need of treatment or that might benefit from such treatment via any suitable route. The preferred route is intravenously.

The actual amount administered, and rate and time-course of administration, will depend on the nature and severity of what is being treated and the precise nature of the form of FVII and FVIIa to be administered. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

For example, in one embodiment, a suitable dose of a Factor VII polypeptide of the invention ranges from about 0.05 mg to 500 mg/day, preferably from about 1 mg to 200 mg/day, and more preferably from about 10 mg to about 175 mg/day for a 70 kg subject as loading and maintenance doses, depending on the weight of the subject and the severity of the condition.

In prophylactic applications, compositions containing a Factor VII polypeptide of the invention are administered to a subject susceptible to or otherwise at risk of a disease state or injury to enhance the subject's own coagulative capability. Such an amount is defined to be a “prophylactically effective dose”. In prophylactic applications, the precise amounts once again depend on the subject's state of health and weight, but the dose generally ranges from about 0.05 mg to about 500 mg per day for a 70-kilogram subject, more commonly from about 1.0 mg to about 200 mg per day for a 70-kilogram subject.

Single or multiple administrations of the compositions can be carried out with dose levels and patterns being selected by the treating physician. For ambulatory subjects requiring daily maintenance levels, the Factor VII variants may be administered by continuous infusion using e.g. a portable pump system.

Local delivery of the Factor VII polypeptide of the present invention, such as, for example, topical application may be carried out, for example, by means of a spray, perfusion, double balloon catheters, stent, incorporated into vascular grafts or stents, hydrogels used to coat balloon catheters, or other well established methods. In any event, the pharmaceutical compositions should provide a quantity of a Factor VII polypeptide sufficient to effectively treat the subject.

Unless otherwise defined, all technical and scientific terms used herein have the meaning commonly understood by a person who is skilled in the art in the field of the present invention.

Preferred features for the second and subsequent aspects of the invention are as for the first aspect mutatis mutandis.

The invention will now be further described by way of reference to the following Examples and Figures which are provided for the purposes of illustration only and are not to be construed as being limiting on the invention. Reference is made to a number of Figures in which:

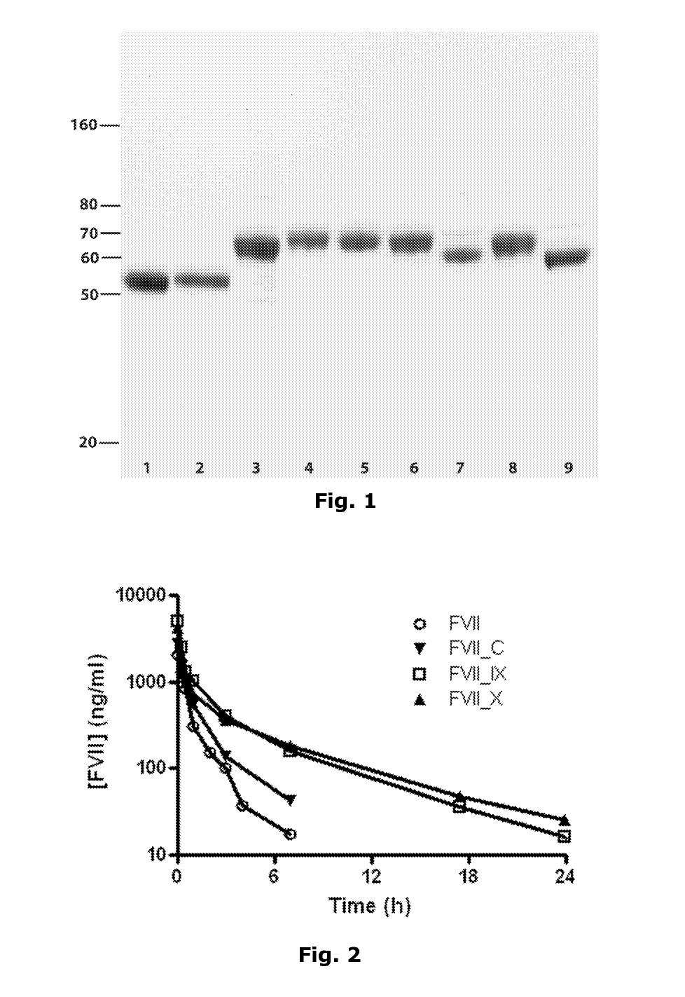

FIG. 1. Purified FVII and FVII variants. Reducing SDS-PAGE showing FVII (lane 1), FVII_C (lane 2), FVII_IX (lane 3), FVII_X (lane 4), FVII_X_{HC} (lane 5), FVII_X_{TTAA} (lane 6), FVII_X_{NNAA} (lane 7), FVII_X₃₀₋₅₂ (lane 8) and FVII_X₁₋₃₄ (lane 9). FVII_X_{HC} is the FVII fusion polypeptide which has the FX activation peptide fused to the C-terminus.

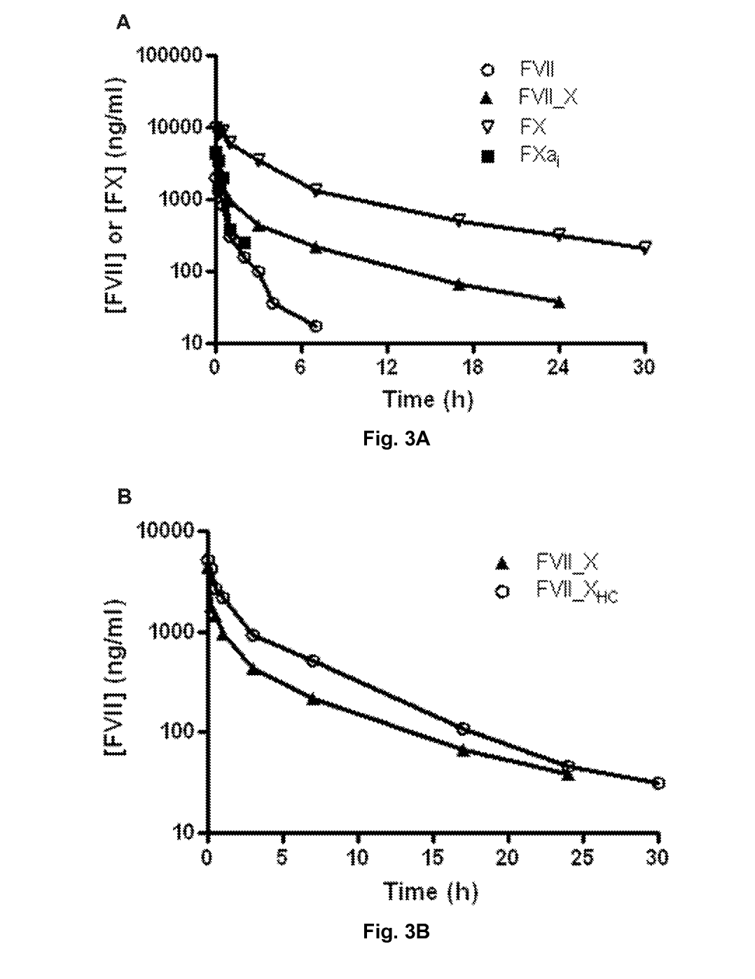

FIG. 2. Pharmacokinetic profiles of FVII variants with different activation peptides. FVII antigen as a function of time after intravenous administration of 0.5-1 mg/kg FVII, FVII_C, FVII_IX and FVII_X (mean, n=1-3).

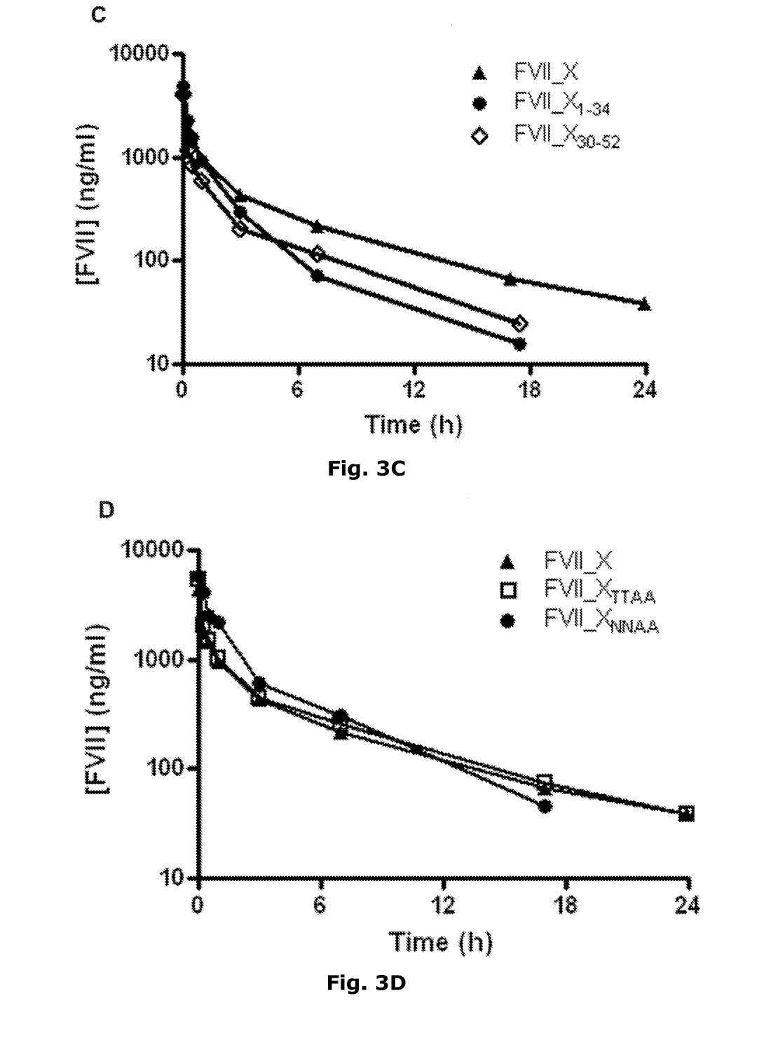

FIG. 3. Effect of FX activation peptide and variants thereof on the half-life of FVII. (A) Plasma concentration of FVII or FX antigen as a function of time after intravenous administration of 1 mg/kg FX, FVII_X, FXaᵢ and FVII to mice (mean, n=1-2). (B) Plasma concentration of FVII antigen as a function of time after intravenous administration of 1 mg/kg FVII_X and FVII_X_{HC}, (C) FVII_X, FVII_X₁₋₃₄ and FVII_X₃₀₋₅₂ and (D) FVII_X, FVII_X_{NNAA} and FVII_X_{TTAA} (mean, n=2-3).

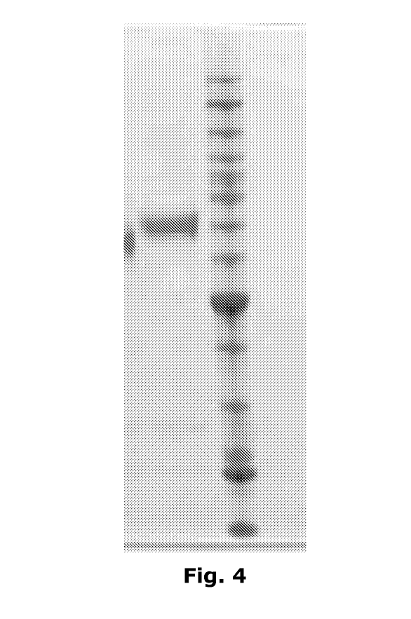

FIG. 4. Non-activation of a variant that has the FX activation peptide inserted between residues 151 and 153. The variant was incubated with 250 nM FIXa for 20 hours and no activation was determined because the variant still runs as a single band (left lane). The molecule marker is shown in the right lane and the band next to the variant band represents a mass of 70 kDa.

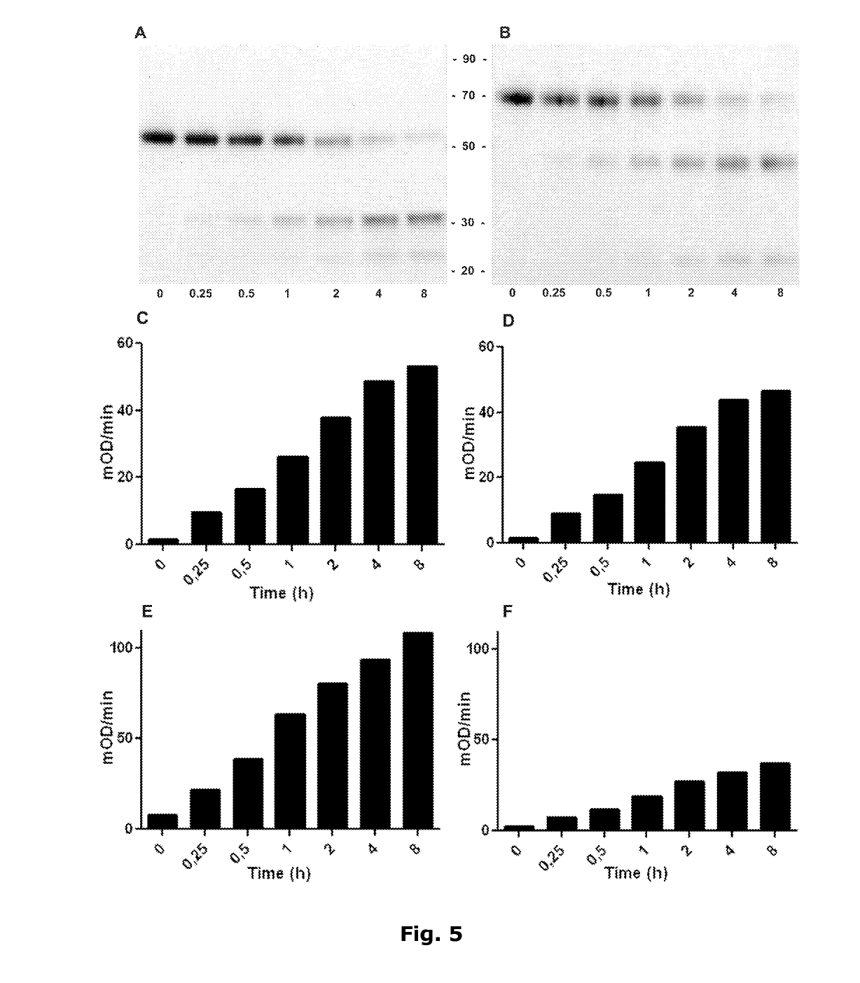

FIG. 5. Activation and enzymatic activity of the FVII_X_{HC} variant. FVII_X_{HC} and FVII were incubated with 80 nM FIXa and analyzed at different time points (0-8 hours). Panels A and B show Coomassie-stained SDS-PAGE of FVII and FVII_X_{HC}, respectively, run under reducing conditions. The amidolytic activity of FVII (C) and FVII_X_{HC} (D) was determined by measuring the turnover of the small chromogenic substrate S-2288. FX was used as substrate to assess the proteolytic activity of FVII (E) and FVII_X_{HC} (F) (mean, n=2).

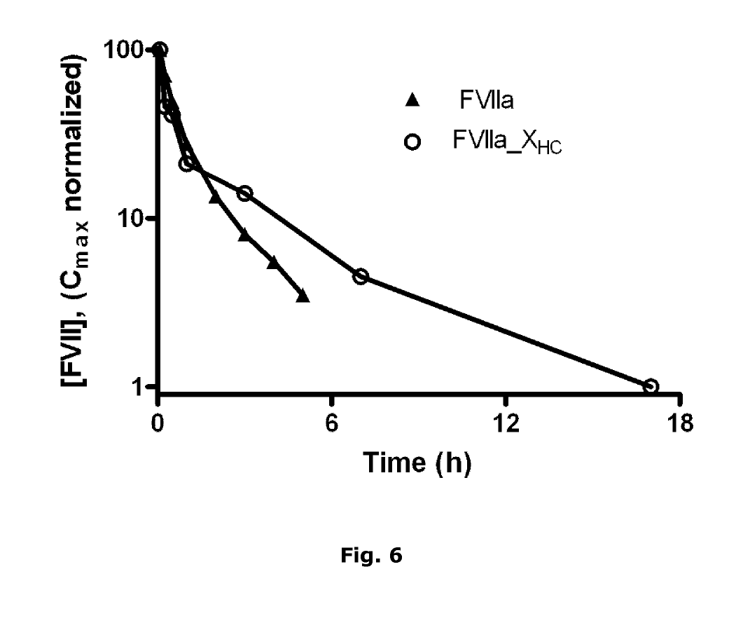

FIG. 6. Pharmacokinetic profiles of FVIIa and FVIIa_X_{HC}. Mean plasma concentration of FVIIa antigen as a function of time (mean, n=2-3) after intravenous administration of 1 mg/kg FVIIa and FVIIa_X_{HC} to mice.

### EXAMPLES

### Materials and Methods

### Reagents

The wild-type FVII expression plasmid pLN174¹⁶ was used as template for the cloning of the FVII mutants. The plasmid pKSLN123 (a gift from Dr. Katrine S. Larsen, Novo Nordisk A/S) was used as template for the PCR of FX activation peptide.

### Construction of FVII Variants

The FVII variant FVII_X, which includes the FX activation peptide between Gly-151 and Ile-153, replacing Arg-152 in FVII, was constructed by overlap extension PCR. In a first PCR reaction, primer pair A and B were used to amplify the FVII light chain, primers C and D the activation peptide to be inserted, and primers E and F to amplify the FVII heavy chain. The sequences of all primers and FVII variants used in this study are found in Tables 1 and 2, respectively. The PCR reaction, using Expand High Fidelity PCR system (Roche), was performed accordingly: after a 4 min heating step at 94° C., 30 cycles of 94° C. 15 s, 55° C. 30 s and 72° C. 2 min followed by an extension time of 7 min at 72° C. The purified PCR products AB and CD were used as templates in a second PCR reaction, carried out as the first, with primer pair A and D to amplify FVII light chain and insert. Subsequently the ABCD and EF products were used as templates with primer pair A and F in a third PCR, where the annealing temperature was raised from 55° C. to 65° C., producing the complete product. Constructs were digested with NheI and NotI restriction enzymes and ligated into a pCI-neo vector (Promega, Madison, Wis., USA).

For the FVII_X_{HC} variant, containing the FX activation peptide in the C-terminal end of FVII, primer pair A and B_X_{HC} was used to amplify FVII, primers C_X_{HC} and D_X_{HC} for FX's activation peptide, followed by a second PCR to generate the whole construct using primers A and D_X_{HC}, otherwise as above. See table 1 for primers.

### Protein Preparation

All FVII variants, including wild-type FVII, were transiently expressed in HEK-293F cells using FreeStyle 293 Expression System (Invitrogen) according to the manual. 96 hours after transfection, the cells were removed by centrifugation and the supernatants saved at −80° C. until use. The expressed proteins were purified by affinity chromatography, using a Ca²⁺-dependent monoclonal anti-FVII antibody, F1A2, coupled to Sepharose.¹⁷ In short, the supernatants were prepared to 350 mM NaCl, 10 mM CaCl₂, pH 7 and loaded on a column, which was equilibrated with 50 mM HEPES, 100 mM NaCl, 10 mM CaCl₂ pH 7.5. The column was washed with 50 mM HEPES, 2 M NaCl, 10 mM CaCl₂ pH 7.5 followed by elution with 50 mM HEPES, 100 mM NaCl, 10 mM EDTA pH 7.5. After elution, 15 mM CaCl₂ was added and the protein solutions stored at −80° C. Protein purity was assessed by Coomassie-stained SDS-PAGE (FIG. 1) and estimated to be more than 95% homogeneous for all preparations.

### Pharmacokinetics

Male NMRI mice (Taconic M&B, Denmark) weighing approximately 25 g were acclimated for at least 7 days at the animal facility at Novo Nordisk A/S, Måløv under standardized conditions 12/12 h light/dark cycle, 21° C., 60% relative humidity, watered and fed ad libitum. The study was performed according to guidelines from the Danish Animal Experiments Council, the Danish Ministry of Justice. Mice were dosed 1 mg/kg, with the exception of FVII_C (0.5 mg/kg), as a single intravenous bolus in the tail vein and blood was obtained according to a sparse sampling design as previously described, including 3 blood samples per mouse and 2 or 3 mice per time point. The mice were anaesthetized by isofluran/O₂/N₂O for blood sampling and 4 droplets of blood was sampled from the orbital plexus using a capillary glass tube at t=0.08, 0.25, 0.5, 1, 2, 3, 4, 5 and 7 h after administration of FVII and FXaᵢ, and at t=0.08, 0.25, 0.5, 1, 3, 7, 17, 24 and 30 h after administration of all other proteins investigated. Blood (45 μL) was immediately transferred to 5 μL, 0.13 M tri-sodium citrate solution and diluted 5 times in 0.01 M sodium phosphate buffer, 0.145 M NaCl, 0.05% Tween 20, 1% BSA, pH 7.6 followed by centrifugation at 4000 g for 5 min at room temperature. The supernatant representing diluted plasma was collected, placed on dry ice and stored at −80° C. until analysis by means of FVII or FX ELISA. Briefly, FVII antigen concentrations were determined by the FVII-ELISA (DakoCytomatio, Dako, Ejby, Denmark). This two-site monoclonal immuno-enzymometric assay with peroxidase as the marker enzyme was performed as described by the manufacturer. FX antigen concentrations were determined by the FX-ELISA by using a modified commercially available kit from Haemochrom Diagnostica (Frederiksberg, Denmark). Briefly, diluted plasma samples were incubated in microwells coated with a polyclonal antibody specific for FX. After a washing step, polyclonal FX antibody coupled to peroxide was added. Following a washing step, a substrate (TMB) in the presence of H₂O₂ was introduced and the colour developed. The reaction was stopped with sulfuric acid, and the amount of colour developed was directly proportional to the concentration of FX in the tested sample. For the initial study, the terminal half-life was estimated by non-compartmental methods (WinNonlin Pro version 4.1 (Pharsight corporation, Mountain View, Calif., USA). For the second study, pharmacokinetic parameters were estimated using a population approach using non-linear mixed effect modelling through 1- or 2-compartmental FOCE method (NON-MEM version VI).²¹ The inter-individual variability was modeled by an exponential error model and the intra-individual variability was modeled as a proportional error model. The quality of the fit was evaluated by graphic analysis of predicted versus observed concentrations, by weighted residuals versus predicted concentrations and by comparison of the objective values.

### Activation and Activity of FVII Variants

In the initial assessment of whether the FVII variants could be activated they were incubated at 1 μM in 50 mM HEPES, 100 mM NaCl, 10 mM CaCl₂, pH 7.4, at ambient temperature with either 250 nM FIXa for 20 h, 100 nM FXa for 2 h, 75 nM FXIa for 2.5 h or 100 nM FVIIa together with 75 nM lipidated TF for 20 h. The samples were analyzed on SDS-PAGE run under reducing conditions. Wild-type FVII was used as a positive control and was completely activated under all described conditions.

FVII_X_{HC} was studied in more detail by incubating 2 μM of FVII or FVII_X_{HC} with 80 nM FIXa in 50 mM HEPES, 100 mM NaCl, 10 mM CaCl₂, 0.01% Tween-80, pH 7.4 at ambient temperature. Samples were taken after 0, 0.25, 0.5, 1, 2, 4 and 8 h and analyzed for amidolytic and proteolytic enzyme activity as well as by SDS-PAGE under reducing conditions. Hydrolysis of chromogenic substrate was monitored at 405 nm using a kinetic microplate reader (SpectraMax 384Plus, Molecular Devices, Sunnyvale, Calif., USA). The amidolytic activity was measured by incubating 12.5 nM FVII/FVII_X_{HC} from the activation mixture with 50 nM sTF and 1 mM S-2288. The proteolytic activity was measured by incubating 100 nM FVII/FVII_X_{HC} plus 500 nM sTF with 150 nM FX for 10 minutes, whereafter the reaction was quenched using excess EDTA and the FXa activity was measured by adding S-2765 (final concentration 0.5 mM). FVIIa_X_{HC} for pharmokinetic analysis was prepared by autoactivation.

### Results

### Activation and Specific Activity of the Activation Peptide-Containing FVII Variants.

For FVII to be converted to its active form, FVIIa, it requires proteolysis of a singe peptide bond between Arg-152 and Ile-153, which is at the position where the activation peptide ends in all but one of the FVII variants (except FVII_X_{HC} where the activation peptide is located C-terminally in FVII). An important question is thus if the variants still can be activated and perform any biological function. Initially, FVII variant activation was tested with the enzymes FVIIa, FIXa and FXa, all physiological activators of FVII. The FVII variants were incubated with high concentrations of the activators for long times which was more than sufficient to completely activate FVII (for details see Materials and Methods). The samples were assayed by reducing SDS-PAGE, where activated proteins would turn up as two bands representing a heavy chain (carrying a C-terminal activation peptide in FVII_X_{HC}) and a light chain (carrying a C-terminal activation peptide in all the other variants). The FVII_X_{HC} variant, containing the FX activation peptide at the C-terminus, was the only variant prone to activation with FVIIa, FIXa and FXa. In all other cases, with the exception of FVII_IX, the variants could not be activated to a detectable extent. FIG. 4 illustrates the results of unsuccessful attempts to activate a FVII variant which has the FX activation peptide inserted between residues 151 and 153 of the FVII polypeptide (after incubation with FIXa for 20 hours). The variant band (left lane in FIG. 4) runs with the same mobility as that of the variant FVII_X_{HC} at time zero in FIG. 5B i.e. it represents uncleaved, intact FVII variant. In contrast, the FVII_X_{HC} variant (i.e. the variant with the activation peptide at the C-terminal) is completely activated after 4 hours incubation with 80 nM FIXa (see FIG. 5B). Activation of FVII_IX was also very slow compared to wild-type FVII and was only seen upon treatment with excessive amounts of FVIIa.

The activation of FVII_X_{HC} was studied in more detail. FVII_X_{HC} was found to exhibit in vitro activation kinetics that were indistinguishable from those of wild-type FVII when incubated with FIXa (FIG. 5A-B) or FXa (not shown). In parallel in the same experiment, the development of catalytic activity of activated FVII_X_{HC} was compared to that of activated FVII (FIG. 5C-F). Activated FVII_X_{HC} displayed almost identical amidolytic activity using the chromogenic substrate S-2288 (FIG. 5C-D). In addition, FVIIa_X_{HC} was able to activate the physiological substrate FX, albeit with an apparent reduction in specific activity compared with FVIIa (FIG. 5E-F).

PK data were also determined as follows. NMRI mice were dosed 1 mg/kg of the listed compounds. The data are set out in table 3. Comp shows the estimated number of compartments in the pharmacokinetic model, Cl is clearance, V₁ is central compartment, V₂ is peripheral compartment, t_{1/2alfa} is the distribution half-life, t_{1/2beta} is disposition/terminal half-life, MRT is mean residence time.

From the data shown in FIG. 6, the terminal half-lives of FVIIa_X_{HC} and FVIIa were estimated to 4.2 h and 1.6 h, respectively, with MRT values of 4.8 and 1.6 h, respectively.

TABLE 1
Sequences of PCR primers

| Primer | Sequence (5′-3′) |
|---|---|
| A | tcactataggctagcatggtctcc |
| B_C | cttggtcttcttggtcttctgtgtcgccttggggtttgctg |
| E_C | accaagaagaccaagtagatccgcgaattgtggggggca |
| B_IX | aaacagtctcagcgccttggggtt |
| C_IX | aaccccaaggcgctgagactgttt |
| D_IX | ccccacaattcgagtgaagtcat |
| E_IX | atgacttcactcgaattgtgggg |
| B_X | tgggccactgagccttggggttt |
| C_X | aaaccccaaggctcagtggccca |
| D_X | ccccacaattcgggtgaggttgtt |
| E_X | aacaacctcacccgaattgtgggg |
| D_X₁₋₃₄ | ccacaattcggtcgaaggggttct |
| E_X₁₋₃₄ | agaaccccttcgaccgaattgtgg |
| B_X₃₀₋₅₂ | aaggggttctcgccttggggttt |
| C_X₃₀₋₅₂ | aaaccccaaggcgagaacccctt |
| F | taaagggaagcggccgcctagggaaat |
| NNAAfwd | gcttgacttcgcccagacgcagcctgagaggggcgacaacgccctcacccg |
| NNAArev | cgggtgagggcgttgtcgcccctctcaggctgcgtctgggcgaagtcaagc |
| TTAAfwd | gacagcatcgcatggaagccatatgatgcagccgacctggaccccgccgagaacc |
| TTAArev | ggttctcggcggggtccaggtcggctgcatcatatggcttccatgcgatgctgtc |
| B_X_{HC} | ctgggccactgagggaaatgg |
| C_X_{HC} | ccatttccctcagtggcccag |
| D_X_{HC} | taaagggaagcggccgcctaggtgaggttg |

TABLE 2
Description of proteins used in current study

| Abbreviation | Protein |
|---|---|
| FVII | Factor VII |
| FVII_C | FVII with protein C activation peptide |
| FVII_IX | FVII with FIX activation peptide |
| FVII_X | FVII with FX activation peptide |
| FVII_X₁₋₃₄ | FVII with the 34 first amino acids of FX activation |
| | peptide |
| FVII_X₃₀₋₅₂ | FVII with the 23 last amino acids of FX activation |
| | peptide |
| FVII_X_{NNAA} | FVII with both N-glycans removed in FX activation |
| | peptide |
| FVII_X_{TTAA} | FVII with both O-glycans removed in FX activation |
| | peptide |
| FVII_X_{HC} | FVII with FX activation peptide inserted in the end of |
| | FVII heavy chain |
| FX | Factor X |
| FXaᵢ | Active site inhibited Factor Xa |
| FVIIa | Factor VIIa |
| FVIIa_X_{HC} | Factor VIIa with FX activation peptide inserted in the |
| | end of FVIIa heavy chain |

TABLE 3
Estimated PK parameters

| | | Dose | Cl | V₁ | V₂ | t_{1/2} _{alfa} | t_{1/2 beta} | MRT |
|---|---|---|---|---|---|---|---|---|
| Compound | Comp | (mg/kg) | (ml/h/kg) | (ml/kg) | (ml/kg) | (h) | (h) | (h) |
| FVII | 2 | 1 | 579 | 301 | 358 | 0.19 | 1.6 | 1.1 |
| FVII_X | 2 | 1 | 176 | 244 | 966 | 0.10 | 6.3 | 6.6 |
| FX | 2 | 1 | 25 | 105 | 102 | 0.08 | 8.6 | 6.3 |
| FXaᵢ | 1 | 1 | 354 | 216 | | | 0.42 | 0.6 |
| FVII_X_{HC} | 2 | 1 | 82 | 194 | 305 | 0.12 | 5.4 | 5.5 |
| FVII_X₁₋₃₄ | 2 | 1 | 249 | 228 | 490 | 0.15 | 3.6 | 2.6 |
| FVII_X₃₀₋₅₂ | 2 | 1 | 295 | 246 | 1080 | 0.13 | 4.2 | 4.4 |
| FVII_X_{NNAA} | 2 | 1 | 115 | 173 | 259 | 0.16 | 3.8 | 3.4 |
| FVII_X_{TTAA} | 2 | 1 | 146 | 200 | 747 | 0.11 | 5.7 | 6.3 |
| FVIIa | — | 1 | — | — | — | — | 1.6 | 1.6 |
| FVIIa_X_{HC} | — | 1 | — | — | — | — | 4.2 | 4.8 |

### SEQUENCE INFORMATION

```
SEQ ID NO: 1
Amino acid sequence of the mature FVII/FVIIa polypeptide.
Ala-Asn-Ala-Phe-Leu-GLA-GLA-Leu-Arg-Pro-Gly-Ser-Leu-GLA-Arg-GLA-Cys-Lys-
                                  5 10 15
GLA-GLA-Gln-Cys-Ser-Phe-GLA-GLA-Ala-Arg-GLA-Ile-Phe-Lys-Asp-Ala-GLA-Arg-
          20 25 30 35
Thr-Lys-Leu-Phe-Trp-Ile-Ser-Tyr-Ser-Asp-Gly-Asp-Gln-Cys-Ala-Ser-Ser-Pro-
                          40 45 50
Cys-Gln-Asn-Gly-Gly-Ser-Cys-Lys-Asp-Gln-Leu-Gln-Ser-Tyr-Ile-Cys-Phe-Cys-
  55 60 65 70
Leu-Pro-Ala-Phe-Glu-Gly-Arg-Asn-Cys-Glu-Thr-His-Lys-Asp-Asp-Gln-Leu-Ile-
                  75 80 85 90
Cys-Val-Asn-Glu-Asn-Gly-Gly-Cys-Glu-Gln-Tyr-Cys-Ser-Asp-His-Thr-Gly-Thr-
                                  95 100 105
Lys-Arg-Ser-Cys-Arg-Cys-His-Glu-Gly-Tyr-Ser-Leu-Leu-Ala-Asp-Gly-Val-Ser-
        110 115 120 125
Cys-Thr-Pro-Thr-Val-Glu-Tyr-Pro-Cys-Gly-Lys-Ile-Pro-Ile-Leu-Glu-Lys-Arg-
                        130 135 140
Asn-Ala-Ser-Lys-Pro-Gln-Gly-Arg-Ile-Val-Gly-Gly-Lys-Val-Cys-Pro-Lys-Gly-
145 150 155 160
Glu-Cys-Pro-Trp-Gln-Val-Leu-Leu-Leu-Val-Asn-Gly-Ala-Gln-Leu-Cys-Gly-Gly-
                165 170 175 180
Thr-Leu-Ile-Asn-Thr-Ile-Trp-Val-Val-Ser-Ala-Ala-His-Cys-Phe-Asp-Lys-Ile-
                                185 190 195
Lys-Asn-Trp-Arg-Asn-Leu-Ile-Ala-Val-Leu-Gly-Glu-His-Asp-Leu-Ser-Glu-His-
        200 205 210 215
Asp-Gly-Asp-Glu-Gln-Ser-Arg-Arg-Val-Ala-Gln-Val-Ile-Ile-Pro-Ser-Thr-Tyr-
                        220 225 230
Val-Pro-Gly-Thr-Thr-Asn-His-Asp-Ile-Ala-Leu-Leu-Arg-Leu-His-Gln-Pro-Val-
235 240 245 250
Val-Leu-Thr-Asp-His-Val-Val-Pro-Leu-Cys-Leu-Pro-Glu-Arg-Thr-Phe-Ser-Glu-
                255 260 265 270
Arg-Thr-Leu-Ala-Phe-Val-Arg-Phe-Ser-Leu-Val-Ser-Gly-Trp-Gly-Gln-Leu-Leu-
                                275 280 285
Asp-Arg-Gly-Ala-Thr-Ala-Leu-Glu-Leu-Met-Val-Leu-Asn-Val-Pro-Arg-Leu-Met-
        290 295 300 305 306
Thr-Gln-Asp-Cys-Leu-Gln-Gln-Ser-Arg-Lys-Val-Gly-Asp-Ser-Pro-Asn-Ile-Thr-
                        310 315 320
Glu-Tyr-Met-Phe-Cys-Ala-Gly-Tyr-Ser-Asp-Gly-Ser-Lys-Asp-Ser-Cys-Lys-Gly-
325 330 335 340
Asp-Ser-Gly-Gly-Pro-His-Ala-Thr-His-Tyr-Arg-Gly-Thr-Trp-Tyr-Leu-Thr-Gly-
                345 350 355 360
Ile-Val-Ser-Trp-Gly-Gln-Gly-Cys-Ala-Thr-Val-Gly-His-Phe-Gly-Val-Tyr-Thr-
                                365 370 375
Arg-Val-Ser-Gln-Tyr-Ile-Glu-Trp-Leu-Gln-Lys-Leu-Met-Arg-Ser-Glu-Pro-Arg-
        380 385 390 395
Pro-Gly-Val-Leu-Leu-Arg-Ala-Pro-Phe-Pro
                        400 405 406
SEQ ID NO: 2
Amino acid sequence of the FIX activation peptide.
Ala-Glu-Ala-Val-Phe-Pro-Asp-Val-Asp-Tyr-Val-Asn-Ser-Thr-Glu-Ala-Glu-Thr-
                                  5 10 15
Ile-Leu-Asp-Asn-Ile-Thr-Gln-Ser-Thr-Gln-Ser-Phe-Asn-Asp-Phe-Thr-Arg
          20 25 30 35
SEQ ID NO: 3
Amino acid sequence of the FX activation peptide.
Ser-Val-Ala-Gln-Ala-Thr-Ser-Ser-Ser-Gly-Glu-Ala-Pro-Asp-Ser-Ile-Thr-Trp-
                                  5 10 15
Lys-Pro-Tyr-Asp-Ala-Ala-Asp-Leu-Asp-Pro-Thr-Glu-Asn-Pro-Phe-Asp-Leu-Leu-
          20 25 30 35
Asp-Phe-Asn-Gln-Thr-Gln-Pro-Glu-Arg-Gly-Asp-Asn-Asn-Leu-Thr-Arg
                  40 45 50 52
```

1. A polypeptide comprising a Factor VII (FVII) or a Factor VIIa (FVIIa) polypeptide or a homologue thereof and a Factor X (FX) or Factor IX (FIX) activation peptide or a homologue thereof, wherein the FX or FIX activation peptide is fused at the C-terminal of the FVII or FVIIa polypeptide.

2. The polypeptide of claim 1, wherein the FVII or FVIIa polypeptide comprises residues 1, 2, 3, 4, 5, 6, 7, 8, 9 or 10 to 397, 398, 399, 400, 401, 402, 403, 404, 405 or 406 of an amino acid sequence as set forth in SEQ ID NO:1 or a homologue thereof, and the activation peptide comprises residues 1, 2, 3, 4, 5 or 6 to 30, 31, 32, 33, 34 or 35 of an amino acid sequence as set forth in SEQ ID NO:2 or a homologue thereof or residues 1, 2, 3, 4, 5 or 6 to 47, 48, 49, 50, 51 or 52 of an amino acid sequence as set forth in SEQ ID NO:3 or a homologue thereof.

3. The polypeptide of claim 1, wherein the FVII or FVIIa polypeptide comprises one or more substitutions relative to the amino acid sequence of SEQ ID NO:1, wherein said substitutions are replacements with any one or more amino acids at a position selected from: 172, 173, 175, 176, 177, 196, 197, 198, 199, 200, 203, 235, 237, 238, 239, 240, 286, 287, 288, 289, 290, 291, 292, 293, 294, 295, 297, 299, 319, 320, 321, 327, 341, 363, 364, 365, 366, 367, 370 or 373 corresponding to amino acid positions of SEQ ID NO:1.

4. The polypeptide of claim 1, wherein the polypeptide is isolated or is recombinant.

5. A nucleic acid sequence encoding the polypeptide of claim 1.

6. A vector comprising the nucleic acid of claim 5.

7. A host cell comprising the nucleic acid of claim 5.

8. A method of producing a FVII polypeptide comprising culturing the host cell of claim 7 under conditions that promote expression of the polypeptide and recovering the expressed polypeptide from the culture.

9. The method of claim 8 further comprising the step of purifying the expressed polypeptide.

10. The method of claim 9, wherein the step of purification includes activating the FVII polypeptide.

11. A pharmaceutical composition comprising the polypeptide of claim 1.

12-14. (canceled)

15. A method of treating a condition associated with a blood clotting deficiency comprising administering the polypeptide of claim 1 to a subject.

16. The method of claim 15, wherein the blood clotting deficiency is haemophilia A, haemophilia B, Factor XI deficiency and Factor VII deficiency, excessive or unwanted bleeding including bleeding in surgery or other tissue damage, or bleeding due to a defective platelet function, thrombocytopenia or von Willebrand's disease.
